(19) United States
(12) Patent Application Publication
(10) Pub. No.: US 2012/0082708 A1
(43) Pub. Date: Apr. 5, 2012

(54) NOVEL METHOD FOR PREPARING COMPOSITE PARTICLE COMPRISING SURFACE TREATMENT LAYER OF SUNSCREENING AGENT FORMED THEREON

(75) Inventors: Sung-ho LEE, Seoul (KR); Jin-kwan PARK, Ansan city (KR); Bou-kun CHOI, Ansan city (KR); Dae-sung BAEG, Ansan city (KR)

(73) Assignee: SUNJIN CHEMICAL CO., LTD., Ansan city (KR)

(21) Appl. No.: 13/238,591

(22) Filed: Sep. 21, 2011

(30) Foreign Application Priority Data
Oct. 4, 2010 (KR) 10-2010-0096309

(51) Int. Cl. A61K 8/49; A61Q 17/04; A61K 8/02

(52) U.S. Cl. 424/401; 424/59

(57) ABSTRACT

The present invention relates to a method of preparing a composite powder comprising a surface treatment layer of sunscreen. The present invention prepares a composite powder comprising a surface treatment layer of sunscreen by a dry process that does not require separate dehydration and drying processes, and thus, the prepared composite powder may be uniformly surface-treated with more quantities of sunscreens compared to a composite powder comprising a surface treatment layer of sunscreen prepared by a conventional wet process, may exhibit remarkably improved sun screen effect, and be useful for a cosmetic composition.

### BACKGROUND OF THE INVENTION

(a) Field of the Invention

The present invention relates to a method of preparing a composite powder comprising a surface treatment layer of sunscreen and exhibiting improved UV blocking effect.

(b) Description of the Related Art

In general, powder, emulsion, water, and the like are used in cosmetics, and the kind and mixing ratio thereof are varied according to use purpose of the cosmetics. Powder which is mainly used for makeup cosmetics for compensating skin defects is largely classified into three types of body pigment for compensating skin defects and improving properties (for example, talc, mica, sericite, silica, and the like), white pigment for providing natural shielding to skin (for example, titanium dioxide, zinc oxide, and the like), and a color pigment for adjusting skin tone.

In general, since a makeup powder product for UV blocking is difficult to contain 10% or more of liquid ingredients, application of organic sunscreen mainly consisting of liquid ingredients is extremely limited. Thus, titanium dioxide powder and zinc oxide powder are predominantly used for UV blocking.

However, recently, talc of which surface is treated with methylene bis-benzotriazolyl tetramethylbutylphenol (MBBT-TALC DM, manufactured by Dai Nippon Kasei Co. Ltd.), which is an organic sunscreen ingredient easily applicable for sunscreen makeup powder, has been developed and commercialized. However, since the powder is manufactured by a wet process wherein all the ingredients should be mixed and reacted in a solution, and then, dehydration and drying are required, the manufacture process is complicated and productivity is low due to high process cost.

### SUMMARY OF THE INVENTION

Accordingly, it is an object of the present invention to provide a method of preparing a composite powder which comprises a surface treatment layer of sunscreen and exhibits improved UV blocking effect, and a composite powder prepared thereby.

It is another object of the present invention to provide a cosmetic composition comprising the composite powder.

The present invention provides a method of preparing a composite powder comprising a surface treatment layer of sunscreen formed thereon, which method comprises dry mixing a powder having hydroxyl group at the end with methylene bis-benzotriazolyl tetramethylbutylphenol so as to prepare a powder of which surface is treated with methylene bis-benzotriazolyl.

The method may further comprise secondarily surface treating the surface treated powder with a surface treating agent selected from the group consisting of alkylsilane, aminosilane, epoxysilane, silicon oil, stearic acid, and a mixture thereof.

The present invention also provides a composite powder prepared by the above method.

The present invention also provides a cosmetic composition comprising the composite powder.

According to the present invention, since a composite powder of which surface is treated with methylene bis-benzotriazolyl tetramethylbutylphenol, which has been conventionally prepared only by a wet process, is prepared by a dry process, a separate process such as dehydration, drying, and the like is not required, and any additional facilities are not required other than a powder mixer, and thus the process may be minimized and productivity may be increased.

And, the composite powder comprising a surface treatment layer of sunscreen prepared by the method of the present invention may exhibit significantly improved UV blocking effect because more MBBT are uniformly treated on the surface of the powder compared to the composite powder of which surface is treated with MBBT by a conventional wet process.

### BRIEF DESCRIPTION OF THE DRAWINGS

FIG. 1 is scanning electron microscope photograph of talc that is primarily surface treated with methylene bis-benzotriazolyltetramethylbutylphenol according to Example 1.

### DETAILED DESCRIPTION OF THE EMBODIMENTS

The term “alkyl” used herein refers to a linear or branched saturated C₁ to C₂₀ hydrocarbon radical chain. Specific examples thereof may include methyl, ethyl, n-propyl, isopropyl, n-butyl, isobutyl, t-butyl, n-pentyl, isopentyl, hexyl, and the like, but not limiter thereto.

The present invention may prepare a composite powder that is surface treated with MBBT by a chemical bond instead of a simple physical mixing, by surface treating the surface of the powder by a dry process wherein a powder and a solid state MBBT are agitated and mixed in a reactor equipped with an agitator, without using a conventional wet process wherein a powder and sunscreen, specifically MMBT are mixed and reacted in a solution state.

Thus, the method of preparing a composite powder comprising a surface treatment layer of sunscreen includes dry mixing a powder having hydroxyl group at the end with MBBT so as to prepare a powder of which surface is treated with MBBT (step 1), and optionally, secondarily surface treating the primarily surface treated powder with a surface treating agent selected from the group consisting of alkyl silane, aminosilane, epoxysilane, silicon oil, stearic acid, and a combination thereof (step 2).

Hereinafter, the method of preparing a composite powder comprising a surface treatment layer of sunscreen according to the present invention will be explained in detail according to each step.

(step 1)

In the step 1, a powder of which surface is treated with methylene bis-benzotriazolyl is prepared by dry mixing.

Specifically, a powder having hydroxyl group at the end and MBBT are introduced in a reactor equipped with an agitator, and then, agitated by rotation of the reactor to dry mix them.

The powder that can be used in the present invention may include any powder used in a cosmetic composition having hydroxyl group (OH) at the end per se or by a separate surface treatment without specific limitation. The surface treatment may be conducted by a common surface treatment method giving a hydroxgy group at the end.

The powder may have a fine particle shape, a flake shape, and the like, and it may be preferable to use two or more kinds of powders having different shapes because it may increase dispersibility of the powder.

If a fine particle powder is used, the particle size may be optionally selected without specific limitation. Specifically, the average particle size may be 0.001 to 1000 μm, preferably 0.001 to 100 μm, more preferably 0.01 to 50 μm, when the particle diameter is measured using a scanning or transmission electron microscope or using a particle size analyzer. If the average particle size of the particle powder is within the above range, coatability of the sunscreen MBBT may be good and use feeling may be good when prepared into cosmetics. Specific examples of the particle powder may include titanium oxide particle, zinc oxide particle, silica particle and a mixture thereof.

Meanwhile, a flake powder may inhibit cohesion of the particle powder and increase dispersibility, and it may include platy powder and scaly powder.

In the flake powder that can be used in the present invention, the thickness to longer diameter may be selected without specific limitations. Specifically, the thickness to longer diameter may be 0.001 to 1000 μm, preferably 0.001 to 50 μm. If the thickness to longer diameter is within the above range, cohesion of the particle powder may be inhibited and the particle powder may be uniformly dispersed. Specific examples of the flake powder may include barium sulfate, silk powder, talc, mica, sericite, kaolin, titanium dioxide coated mica, iron oxide coated mica, chromium oxide coated mica, calcium carbonate, magnesium carbonate, aluminum silicate, magnesium sulfate, and a mixture thereof.

The sunscreen methylene bix-benzotriazolyl tetramethylbutylphenol (MBBT) may be prepared by a common method or commercially obtained.

The MBBT may be used in an amount of 0.5 to 50 parts by weight, preferably 0.5 to 30 parts by weight, based on 100 parts by weight of the powder. If the MBBT is used within the above range, UV blocking effect may be maintained without hindering adhesion of the powder to skin as cosmetic raw material, and modified powder may be maintained smooth.

The reaction temperature at which the powder is surface treated with MBBT may be appropriately selected according to the kind of raw material and the particle diameter of the raw material, and it may be preferable to gradually raise the temperature from the initial mixing temperature to a selected suitable temperature of 80 to 200° C. More preferably, it may be conducted at a temperature range of from 80 to 130° C. If the reaction temperature is within the above range, finally prepared composite powder may exhibit excellent water repellency and UV blocking effect. According to the present invention, although a melting point of MBBT is about 196° C. which is higher than the above determined temperature, MBBT is coated on the surface of powder by friction and heat generated during agitation mixing.

Further, the MBBT surface treatment may be preferably conducted with agitation while observing the temperature and color and checking water repellency and use feeling, wherein the agitation may be conducted at 100 rpm or more, preferably 500 to 5,000 rpm, more preferably 1,000 to 3,000 rpm. If the agitation speed is within the above range, finally prepared UV blocking composite powder may have excellent coatibility, i.e., excellent water repellency, thus having good use feeling when used as cosmetics.

Further, the MBBT surface treatment may be preferably conducted while gradually supplying steam in order to further accelerate friction heat to reduce time until the surface treatment temperature is reached and increase surface treatment efficiency.

(Step 2)

Step 2 is an optional step wherein the powder that is surface treated with MBBT in the step 1 is secondarily surface treated by introducing a surface treating agent in the same reactor with agitation.

Specific examples of the surface treating agent may include C1-20 alkyl silane, aminosilane, epxoysilane, silicon oil (for example, dimethicone, methicone, cyclomethicone, and the like), stearic acid, and a mixture thereof. By the secondary surface treatment with the above surface treating agent, the composite powder may exhibit excellent water repellency applicable for cosmetics, and discoloration due to exposure of MBBT to UV may be prevented when applied for cosmetics.

The surface treating agent may be used in an amount of 0.2 to 20 parts by weight, preferably 1 to 5 parts by weight, based on 100 parts by weight of the powder. If it is used within the above range, excellent water repellency may be obtained. If the amount of the surface treating agent is less than 0.2 parts by weight, water repellency may be too low and thus the powder may be inappropriate for cosmetics, and if it exceeds 20 parts by weight, unique use feeling of the powder may be deteriorated when applied for cosmetics.

When secondary surface treatment is conducted with the surface treating agent, the agitation speed and temperature are not specifically limited, but it may be preferably conducted at 1000 to 200 rpm and 60 to 150° C.

The process of preparing a composite powder that is surface treated with sunscreen is conducted in one reactor. The reactor that can be used is not specifically limited as long as it may provide sufficient friction or heat enabling reaction of powder and MBBT through agitation, and preferably, it may be further equipped with a heating apparatus so as to rapidly increase temperature in the reactor. The agitator and the heating apparatus may be equipped in the reactor, or separately attached to the reactor. Specific examples of the reactor may include a Henschel mixer, a reactor dryer, and other container capable of agitating powder, and the like.

According to the method of the present invention, a separate drying process is not required because moisture content in the finally prepared composite powder is less than 1.0 wt %, and since surface treatment is conducted by a dry process, more MBBT may be uniformly treated on the surface of powder compared to the conventional wet process, thus exhibiting excellent UV blocking effect. As the result, a composite powder comprising a surface treatment layer of sunscreen prepared according to the present invention may be useful for a cosmetic composition.

Therefore, the present invention also provides a UV blocking composite powder prepared according to the above method, and a cosmetic composition comprising the same.

Examples of the applicable cosmetics may include powder type cosmetics such as powder foundation, white powder, or face powder; and emulsified liquid type cosmetics such as sun cream or cream, and the examples of the cosmetics wherein the effect of the present invention may be remarkably displayed may include functional cosmetics having UV blocking function such as UV blocking powder foundation, UV blocking white powder, UV blocking BB cream, or sun cream.

The content of the composite powder included in the cosmetic composition may be appropriately controlled according to the properties of the products, and preferably, it may be included in the content of 0.5 to 50 wt %, based on total weight of the cosmetic composition. If it is included within the above range, properties of cosmetics and use feeling may be simultaneously satisfied.

The cosmetic composition according to the present invention may further include powders, a colorant, an emulsion, a moisturizer, a surfactant, a sterilizer, flavorings, a solvent, salts, viscous material, polymer, antiseptics, commonly used in powder cosmetics, in addition to the UV blocking composite powder.

Hereinafter, the present invention will be explained in more detail with reference to the following examples. However, these examples are only to illustrate the invention and the scope of the invention is not limited thereto.

### Example 1

7.8 kg of talc (JA-46R, manufactured by Asada Milling Co., Ltd) was introduced in a Henschel mixer, and then, 2 kg of methylene bis-benzotriazolyl tetramethylbutylphenol was introduced, and agitated at high speed of 2500 rpm to prepare talc of which surface is primarily treated with methylene bis-benzotriazolyl tetramethylbutylphenol. During the agitation, steam was slowly introduced to raise the temperature to 100° C., and temperature and color of the reactant were observed and water repellency and use feeling were checked.

After primary surface treatment was completed, 0.2 kg of dimethicone was added to the surface treated talc, and agitated at a high speed of 1500 rpm to conduct secondary surface treatment. The finally produced composite powder had very excellent water repellency, and moisture content of 0.8 wt %.

### Example 2

8.0 kg of mica (MC0800, manufactured by MERCK) was introduced in a Henschel mixer, and then, 1.5 kg of methylene bis-benzotriazolyl tetramethylbutylphenol was introduced and agitated at a high speed of 2000 rpm to prepare mica of which surface is primarily treated with methylene bis-benzotriazolyl tetramethylbutyl phenol. During the agitation, steam was slowly introduced to raise the temperature to 80° C., the temperature and color of the reactant were observed, and water repellency and use feeling were checked.

After the primary surface treatment was completed, 0.5 kg of methicone was added to surface treated mica, and then, agitated at a high speed of 1500 rpm to conduct secondary surface treatment. The finally produced product has very excellent water repellency, and moisture content of 0.8 wt %.

### Example 3

8.0 kg of sericite (B-PAS1000, manufactured by KAOLIN company) was introduced in a Henschel mixer, and then, 1.5 kg of methylene bis-benzotriazolyl tetramethylbutylphenol was introduced and agitated at a high speed of 2000 rpm to prepare sericite of which surface is primarily treated with methylene bis-benzotriazolyl tetramethylbutyl phenol. During the agitation, steam was slowly introduced to raise the temperature to 80° C., the temperature and color of the reactant were observed, and water repellency and use feeling were checked.

After the primary surface treatment was completed, 0.5 kg of methicone was added to surface treated sericite, and then, agitated at a high speed of 1000 rpm to conduct secondary surface treatment. The finally produced product has very excellent water repellency, and moisture content of 0.6 wt %.

### Example 4

A cosmetic composition was prepared according to the composition and the contents as described in the following Table 1.

TABLE 1

| | | content |
|---|---|---|
| No | Ingredient | (parts by weight) |
| 1 | Composite powder of Example 1 | 20.0 |
| 2 | Mica that is surface treated with alkylsilane | 25.0 |
| | (SUNMICA-AS, manufactured by Sunjin | |
| | Chemical Co. Ltd.) | |
| 3 | Sericite that is surface treated with | 28.0 |
| | alkylsilane | |
| | (SUNSERI-AS, manufactured by Sunjin | |
| | Chemical Co. Ltd.) | |
| 4 | Zinc stearate | 3.0 |
| 5 | Silica (SUNSIL-130, manufactured by | 6.0 |
| | Sunjin Chemical Co. Ltd.) | |
| 6 | Titanium dioxide of white pigment grade | 8.0 |
| | (SUNTITAN-AS, manufactured by Sunjin | |
| | Chemical Co. Ltd.) | |
| 7 | Octylmethoxy cinnamate | 5.0 |
| | (PARSOL MCX, manufactured by DSM | |
| | Nutritional Products) | |
| 8 | Dimethicone (KF96-100cs, manufactured | 5.0 |
| | by Shinetsu Chemical Co. Ltd.) | |

### Comparative Example 1

A cosmetic composition was prepared by the same method as Example 4, except using talc (MBBT-TALC DM, manufactured by Dai Nippon Kasei Co. Ltd.) of which surface is primarily surface treated with MBBT and then secondarily treated with dimethicone by a conventional wet process instead of the composite powder prepared in Example 1.

### Experimental Example 1

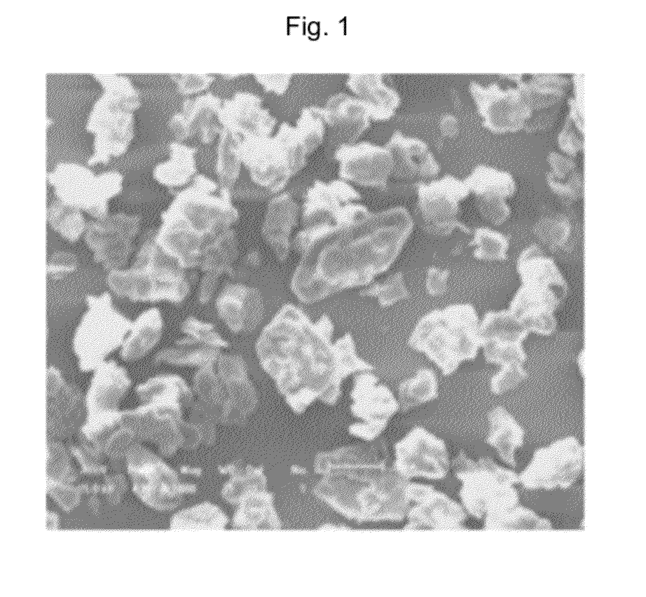

When a composite powder was prepared according to Example 1, talc that is primarily surface treated with MBBT was observed by scanning electron microscope (magnification ×1,000). The result is shown in FIG. 1.

As shown in FIG. 1, MBBT is uniformly coated on the surface of powder.

### Experimental Example 2

For the composite powder prepared in Example 1 and the conventional composite powder used in Comparative Example 1 (MBBT-TALC DM, Dai Nippon Kasei Co. Ltd.), the content of organic sunscreen ingredient of methylene bis-benzotriazolyl tetramethylbutylphenol that is surface-treated on the surface of each powder is quantitatively analyzed using High-performance liquid chromatography (HPLC), and the results is shown in Table 2.

TABLE 2

| | | Conventional |
|---|---|---|
| | Composite | composite |
| | powder of | powder used in |
| | Example 1 | Comparative Example 1 |
| Methylene | 19.4% | 11.8% |
| bis-benzotriazolyl | | |
| tetramethylbutylphenol | | |
| (wt %) | | |

As shown in Table 2, the composite powder of Example 1 prepared according to the present invention has about 10 wt % higher content of an organic sunscreen ingredient of methylene bis-benzotriazolyl tetramethylbutylphenol than conventional composite powder (MBBT-TALC DM, manufactured by Dai Nippon Kasei Co. Ltd.) used in Comparative Example 1. Thus, it can be expected that the composite powder prepared according to the present invention exhibits more excellent UV blocking performance

### Experimental Example 3

To confirm UV blocking effect of the composite powder of the present invention, UV-B blocking effects and UV-A blocking effects of the cosmetic compositions prepared in Example 4 and Comparative Example 1 were measured using optometric 290 (In-vitro SPF analyzer), and respectively indicated as SPF (Sun Protection Factor) index and UV-A ratio. The results are shown in Table 3. The SPF index and UV-A ratio were measured total 6 times, and the average values are described.

TABLE 3

| | Example 4 | Comparative Example 1 |
|---|---|---|
| SPF index | 21.4 | 16.2 |
| UV-A ratio | 0.79 | 0.61 |

As shown in Table 3, the cosmetic composition of Example 4 comprising the composite powder prepared according to the present invention exhibits about 30% or more excellent UV-B and UV-A blocking effects than the cosmetic composition of Comparative Example 1.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A method of preparing a composite powder comprising a surface treatment layer of sunscreen formed thereon, which method comprises dry mixing a powder having hydroxyl group at the end with methylene bis-benzotriazolyl tetramethylbutylphenol so as to prepare a powder of which surface is treated with methylene bis-benzotriazolyl.

2. The method according to claim 1, wherein the methylene bis-benzotriazolyl tetramethylbutylphenol is included in an amount of 0.5 to 50 parts by weight, based on 100 parts by weight of the powders.

3. The method according to claim 1, wherein the surface treatment is conducted with raising the reaction temperature.

4. The method according to claim 1, wherein the surface treatment is conducted at a temperature range of from 80 to 200° C.

5. The method according to claim 1, wherein the powder is selected from the group consisting of titanium oxide powder, zinc oxide powder, silica powder, barium sulfate, silk powder, talc, mica, sericite, kaolin, titanium dioxide coated mica, iron oxide coated mica, chromium oxide coated mica, calcium carbonate, magnesium carbonate, aluminum silicate, magnesium sulfate, and a mixture thereof.

6. The method according to claim 1, further comprising secondarily surface treating the surface treated powder with a surface treating agent selected from the group consisting of alkyl silane, aminosilane, epoxysilane, silicon oil, stearic acid, and a mixture thereof.

7. The method according to 6, wherein the surface treating agent is selected from the group consisting of C1-20 alkylsilane, aminosilane, epoxysilane, silicon oil, stearic acid, and a mixture thereof.

8. The method according to 6, wherein the surface treating agent is included in an amount of 0.2 to 20 parts by weight, based on 100 parts by weight of the powders.

9. A composite powder comprising a surface treatment layer of sunscreen, which is prepared by the method according to claim 1.

10. A cosmetic composition comprising the composite powder according to claim 9.
